(19) United States
(12) Patent Application Publication
(10) Pub. No.: US 2022/0315955 A1
(43) Pub. Date: Oct. 6, 2022

(54) CARBON NEGATIVE PRODUCTION OF DIACIDS AND OTHER BIOCHEMICALS USING CELL FREE BIOCATALYSIS

(71) Applicant: Alliance for Sustainable Energy, LLC, Golden, CO (US)

(72) Inventors: Yannick J. BOMBLE, Arvada, CO (US); Petri Markus ALAHUHTA, Pine, CO (US); Simon James Bradshaw MALLINSON, Denver, CO (US); Michael E. HIMMEL, Littleton, CO (US)

(21) Appl. No.: 17/713,015

(22) Filed: Apr. 4, 2022

Related U.S. Application Data
(60) Provisional application No. 63/170,367, filed on Apr. 2, 2021.

(51) Int. Cl. C12P 7/52

(52) U.S. Cl. CPC C12P 7/52

(57) ABSTRACT

Disclosed herein is a novel, cofactor balanced, cell-free biocatalysis pathway to make diacids from formaldehyde or methanol that does not use ATP and produces no CO₂. In another embodiment, disclosed herein is a novel C5/C6 (hydrolysate) utilization pathway that interplays with the cell making diacids from formaldehyde or methanol that does not use ATP, produces no CO₂, and replaces ATP with (cheap) polyphosphate (no other cofactors needed). Using methods and compositions disclosed herein, no CO₂ is produced and the pathways disclosed herein are cell-free. The combination of pathways, feedstocks and products disclosed herein is novel.

### CROSS-REFERENCE TO RELATED APPLICATIONS

This application claims priority under 35 U.S.C. § 119 to U.S. provisional patent application No. 63/170,367 filed on 2 Apr. 2021, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Harnessing grid electrons in the form of chemical bonds could generate close to 5 Quadrillion BTU of energy, while enabling the flexible use of intermittent sources of renewable energy. Through recent advances in cell-free biocatalysis, novel metabolic pathways, and electrocatalytic reactor design, the dream of fixing CO₂ with low-cost electricity and co-producing biochemicals is within reach.

Regions in the U.S. with high intermittent renewables utilization have started experiencing low and even negative energy rates during times of the day when supply exceeds demand. An approach to avoid expensive storage of energy is to use such curtailed electricity to fix CO2 into chemical intermediates, such as methanol, that can later be upgraded to a variety of biochemicals. Electrocatalytic reduction of CO₂ to chemicals followed by downstream biological upgrading has been explored previously. However, microbial cellular strategies remain hindered by low productivity due in part to substrate and product toxicity. Additionally, CO₂ evolution during microbial conversion of any feedstock to biochemicals remains an obstacle to further decarbonize the bioeconomy. To circumvent CO₂ evolution, several new metabolic pathways have been developed and show promise in vivo. However, these processes would ultimately suffer from low productivity and titers in the production of toxic compounds, such as diacids.

### SUMMARY

In an aspect, disclosed herein is a method for the ATP-independent and carbon neutral/negative cell-free conversion of formaldehyde or methanol to diacids via non oxidative glycolysis, reverse beta-oxidation and omega oxidation comprising the step of contacting a non-naturally occurring cell with formaldehyde or methanol. In an embodiment, there is no CO₂ production. In an embodiment, there is net negative CO₂ production. In an embodiment, the conversion is co-factor balanced. In an embodiment, the co-factor is selected from the group consisting of NADH and NAD+. In another embodiment, no ATP is needed for the cell-free conversion.

In another aspect, disclosed herein is an ATP-independent and carbon neutral/negative cell-free C5/C6 hydrolysate utilization pathway engineered to make diacids via acetyl-CoA. In an embodiment, the hydrolysate is mixed with methanol. In an embodiment, there is no CO₂ production. In an embodiment, there is net negative CO₂ production. In an embodiment, the conversion is co-factor balanced. In an embodiment, the co-factor is selected from the group consisting of NADH and NAD+. In another embodiment, no ATP is needed for the cell-free conversion.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts an overall concept scheme to fix CO₂ and avoid CO₂ evolution for the production of various biochemicals. 1) CO₂ is electrocatalytically reduced to methanol (outside the scope of this proposal). 2) Formaldehyde is converted to acetyl-CoA using novel enzymes to drive formaldehyde condensation. 3) A novel, ATP-independent, C5/C6 non oxidative glycolysis (NOG) pathway is used to generate additional acetyl-CoA intermediates 4) A reverse beta oxidation can be used to produce diacids.

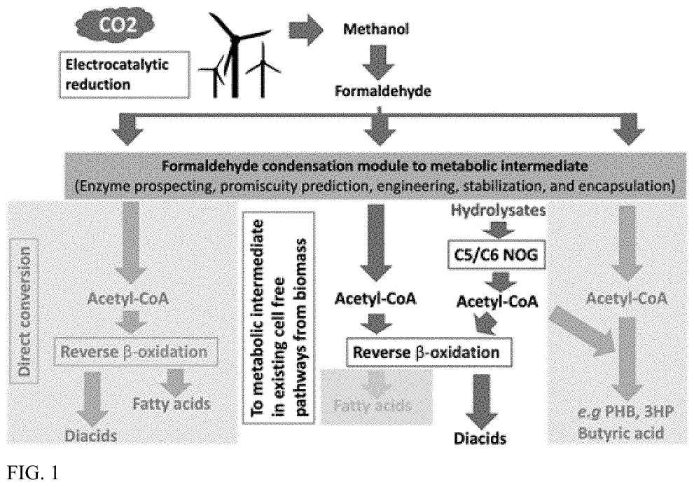

FIGS. 2A, 2B, 2C and 2D depict the details of cell free pathways used to enable the CO₂ negative production of adipic acid. FIG. 2A, formaldehyde condensation module using an engineered glycolaldehyde synthase or novel enzymes. FIG. 2B, novel C5/C6 non oxidative glycolysis FIG. 2C, central intermediate acetyl-CoA from C5/C6 sugars and methanol FIG. 2D, conversion of acetyl-CoA to adipic acid using reverse β oxidation and omega oxidation. Cofactors are recycled with the use of the initial methanol dehydrogenase with no additional sacrificial substrate.

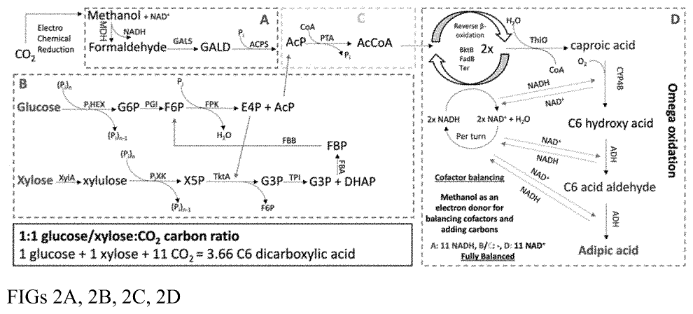

FIG. 3 depicts embodiments of enzyme and metabolic pathway engineering, stabilization and optimization disclosed herein.

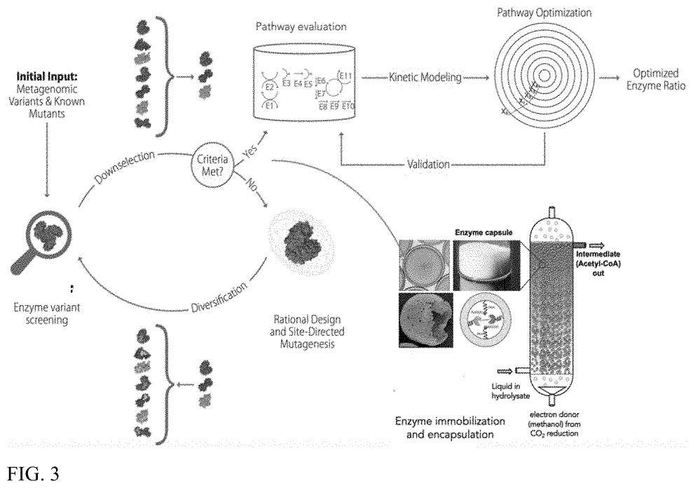

### DETAILED DESCRIPTION

Disclosed herein are methods and compositions of matter for the circumvention of the toxicity bottleneck associated with in vivo systems and further mitigation of CO₂ evolution. In an embodiment, this will be achieved by cell-free biocatalysis concept built on pathways engineered to conserve carbon and efficient formaldehyde condensation to enable conversion of formaldehyde to diacids to further increase carbon efficiency. As disclosed herein, hydrolysates will be used with the incorporation of CO₂-derived methanol. The cell free biocatalysis for mitigating solvent tolerance and use at larger scale has been demonstrated. Additionally, cell free approaches can function by directly plugging into current electrocatalytic unit operations used for CO₂ reduction (i.e., using a “side saddle” reactor). Full-scale employment of this approach offers the potential to use intermittent renewable energy sources during non-peak usage to fix CO₂ in key biopolymer precursors, e.g adipic acid. This technology has the potential to significantly reduce emissions during the production of biochemicals due to the large market share of these key precursors.

Methods and compositions of matter disclosed herein include at least three steps: 1) ATP-independent conversion of CO₂ derived methanol to balance cofactors and produce the key metabolic intermediate, acetyl-CoA, 2) the stabilization and encapsulation of key metabolic enzymes to shield chemical toxicity and enable longer operating lifetime and process scaling, and 3) the carbon negative, ATP-independent production of diacids from multiple substrates combining pathways with no CO₂ evolution. Our approach circumvents limitations that have stymied previous attempts to achieve carbon efficient biochemicals production from electricity and CO₂. The novel combination of efficient metabolic pathways for cell free biocatalysis and the enhanced biocatalyst robustness to substrate and product toxicity are expected to dramatically enhanced productivity, yields, titers, and purity of final products. Eventually, this approach has the potential to disrupt the current bioproducts paradigm, simultaneously fixing CO₂ into a versatile, high energy-density bioproduct and enabling greater industrial adoption of renewable biochemicals by valorizing electricity production.

In an embodiment, disclosed herein are a set of metabolic pathways that are cofactor balanced and allow the carbon neutral production of diacids by combining the oxidative glycolysis (NOG), reverse beta-oxidation and omega oxidation as well as enzymes that can use polyphosphate instead of ATP. This combination with engineered enzymes able to convert formaldehyde to acetyl-COA allow the direct conversion of CO₂ derived methanol/hydrolysate to diacids. These pathways are especially well suited for application in cell free biocatalysis. Eliminating CO₂ evolution during the production of biochemicals is difficult to achieve in microbial systems. Additionally, the utilization of CO₂ directly in microbes is difficult to achieve due to slow transport. Finally, the production of diacids and other toxic chemicals is very limited in classical fermentations. As an example, the methods disclosed herein have at least the following features: ATP-independent and carbon neutral/negative cell-free conversion of formaldehyde or methanol to diacids via non oxidative glycolysis (NOG), reverse beta-oxidation and omega oxidation; and novel ATP-independent and carbon neutral/negative (when mixed with methanol) cell-free C5/C6 (hydrolysate) utilization pathway to diacids via acetyl-CoA that plugs into the main pathway.

In an embodiment, the ATP-independent and carbon neutral/negative cell-free conversion of formaldehyde or methanol to diacids via non oxidative glycolysis (NOG), reverse beta-oxidation and omega oxidation disclosed herein includes a novel combination of known (natural) enzyme reactions/pathways to make diacids (like adipic acid) from formaldehyde or methanol; and no CO₂ production; in an embodiment, can be carbon negative if formaldehyde or methanol from CO₂, uses a co-factor (NADH/NAD+) that is balanced if methanol and formaldehyde are used to make adipic acid at 1:1 methanol:formaldehyde ratio.

Furthermore, in an embodiment, no ATP needed for the reactions disclosed herein. In an embodiment, disclosed herein is a novel ATP-independent and carbon neutral/negative (when mixed with methanol) cell-free C5/C6 (hydrolysate) utilization pathway to diacids via acetyl-CoA that plugs into a main pathway. In another embodiment, disclosed herein is a novel C5/C6 (hydrolysate) utilization pathway of known (natural) enzyme reactions that plugs into an ATP-independent and carbon neutral/negative cell-free conversion of formaldehyde or methanol to diacids via non oxidative glycolysis (NOG), reverse beta-oxidation and omega oxidation.

In an embodiment, no CO₂ is produced if formaldehyde or methanol from CO₂ is used together with hydrolysate. In an embodiment, the reactions disclosed herein can be carbon negative if formaldehyde or methanol from CO₂ is used together with hydrolysate. In another embodiment, co-factor (NADH/NAD+) is balanced and carbon negative if both methanol (from CO₂) and hydrolysate are used to make adipic acid at 1:1 hydrolysate (methanol/formaldehyde) ratio. In another embodiment, no ATP needed because one ATP-dependent kinase is replaced by a polyphosphate dependent kinase which may need supplemental polyphosphate as feedstock.

Disclosed herein are methods and engineered cells for the production of adipic acid from hydrolysates using methanol as an electron donor for cofactor recycling and additional incorporation of carbons in fixed bed flow reactor modules operating at 2 g/L/h continuously over 1 day, producing a final product titer of at least 48 g/L of adipic acid with less than 1% cofactor loss. The ATP-independent cell-free pathway for the conversion of glucose/xylose and methanol will combine 1) an engineered C5/C6 NOG pathway, 2) a reverse beta oxidation pathway, 3) an engineered pathway for the condensation of formaldehyde to acetyl-CoA, and 4) engineered thioesterases able to cleave fatty acids at the desired chain length followed by enzymatic upgrading to corresponding diacids using omega oxidation. Many of these enzymes will be selected from diversity searches to find the most stable enzymes, especially those robust to methanol, formaldehyde, and diacids. To further improve productivity, enzymes will undergo several rounds of engineering in addition to enzyme immobilization or encapsulation to further increase stability and mitigate chemical toxicity.

In an embodiment, methods and genetically engineered organisms are disclosed which embody, identify or engineer enzyme variants capable of tolerating >10 g/L methanol/formaldehyde and 48 g/L of adipic acid. A large metagenomic space will be searched by leveraging the vast enzyme and isolate collections. In an embodiment structure guided rational enzyme engineering will be used to further improve the efficiency of these enzymes. In an embodiment enzyme classes will be identified, and engineer enzyme variants made that are capable of efficiently conducting formaldehyde condensation to glycolaldehyde or other acetyl-CoA intermediates. In an embodiment, a combination of rational design and guided site-specific saturation mutagenesis will be used to improve the promiscuous activity of these enzyme targets. In an embodiment, machine learning approaches will be used to predict potential enzyme promiscuities towards these reactions. In another embodiment, the cell-free pathway will be configured to achieve 2 g/L/h productivity, with consistent operation over several days. A kinetic model of the pathway will be created and parameterized using experimental data, and iteratively optimize by training on experimental results.

In an embodiment, multi-enzyme and cofactor assemblies will be engineered to enable efficient multi-site reactions and cofactor generation. Enzyme co-localization greatly increases reaction efficiency by mitigating the loss of reaction intermediates through diffusion and efficient regeneration of the cofactor are critical due to their high cost. In an embodiment, surface conjugation and porous material synthesis will be used to develop a biomimetic, microcapsule-based multienzyme cascade system to protect intermediate loss and enhance enzyme robustness. In an embodiment, the regeneration of cofactors on conductive surfaces as a means to supplement the enzymatic cofactor recycling will be used as a way to reduce the use of methanol. In another embodiment, the direct electron injection in several redox enzymes will be used to solve pathway redox challenges for longer chain diacids.

Key technoeconomic considerations include electrocatalytic reactor cost, enzyme cost, cofactor cost, and material costs for encapsulation. In an embodiment, cofactors are stable in certain buffers for more than 60 days with no losses. Additionally, enzymatic cofactor recycling with close to >99% recycling efficiency for more than 3 days will be used, indicating that 30+ day cycles are most likely achievable.

In an embodiment, the feedstock used is corn stover hydrolysate. In an embodiment, the reducing equivalent is mainly methanol from CO₂ reduction to balance the cofactors and funnel all carbons to Acetyl-CoA to supplement the hydrolysate. In an embodiment a low amount of phosphite is used for finer control of cofactor balancing if needed with a phosphite dehydrogenase. In an embodiment, the product of the non-naturally occurring cells is adipic acid.

The processes disclosed herein might require significant energy input for the production of methanol from CO₂ but will allow the conversion of all carbons in the main feedstock and from CO₂. If renewable electrons can be used in this process it would enable the cost efficient and clean fixation of CO₂ in products. Additionally, given the market share of the product being targeted it is anticipated that this process could have significant impact on emissions and disrupt the current mode of production of adipic acids and other diacids.

The foregoing disclosure has been set forth merely to illustrate the invention and is not intended to be limiting.

We claim:

1. A method for the carbon neutral cell-free conversion of formaldehyde or methanol to diacids via non oxidative glycolysis, reverse beta-oxidation or omega oxidation comprising the step of contacting a non-naturally occurring cell with formaldehyde or methanol.

2. The method of claim 1 wherein there is no CO₂ production.

3. The method of claim 1 wherein the conversion is co-factor balanced.

4. The method of claim 3 wherein the co-factor is selected from the group consisting of NADH and NAD+.

5. The method of claim 1 wherein no ATP is needed for the cell-free conversion.

6. A method for the carbon negative cell-free conversion of formaldehyde or methanol to diacids via non oxidative glycolysis, reverse beta-oxidation or omega oxidation comprising the step of contacting a non-naturally occurring cell with formaldehyde or methanol.

7. The method of claim 6 wherein there is net negative CO₂ production.

8. The method of claim 6 wherein the conversion is co-factor balanced.

9. The method of claim 8 wherein the co-factor is selected from the group consisting of NADH and NAD+.

10. The method of claim 6 wherein no ATP is needed for the cell-free conversion.

11. A non-naturally occurring organism capable of the carbon neutral cell-free conversion of formaldehyde or methanol to diacids via non oxidative glycolysis, reverse beta-oxidation or omega oxidation.

12. The non-naturally occurring organism of claim 11 wherein there is no CO₂ production.

13. The non-naturally occurring organism of claim 11 wherein the conversion is co-factor balanced.

14. The non-naturally occurring organism of claim 13 wherein the co-factor is selected from the group consisting of NADH and NAD+.

15. The non-naturally occurring organism of claim 11 wherein no ATP is needed for the cell-free conversion.

16. The non-naturally occurring organism of claim 11 capable of the carbon negative cell-free conversion of formaldehyde or methanol to diacids via non oxidative glycolysis, reverse beta-oxidation or omega oxidation.

17. The non-naturally occurring organism of claim 16 wherein there is net negative CO₂ production.

18. The non-naturally occurring organism of claim 16 wherein the conversion is co-factor balanced.

19. The non-naturally occurring organism of claim 18 wherein the co-factor is selected from the group consisting of NADH and NAD+.

20. The non-naturally occurring organism of claim 16 wherein no ATP is needed for the cell-free conversion.
